(19) United States
(12) Patent Application Publication
(10) Pub. No.: US 2023/0400461 A1
(43) Pub. Date: Dec. 14, 2023

(54) METHODS, ANTIBODIES OR ANTIGEN BINDING FRAGMENTS AND OTHER TOOLS FOR DETERMINING TARGET MOLECULES

(71) Applicant: TEKNOLOGIAN TUTKIMUSKESKUS VTT OY, Espoo (FI)

(72) Inventors: Tarja NEVANEN, Espoo (FI); Sirpa JYLHÄ, Espoo (FI); Henri AROLA, Espoo (FI)

(21) Appl. No.: 18/271,140

(22) PCT Filed: Jan. 7, 2022
(86) PCT No.: PCT/FI2022/050014

§ 371 Date: Jul. 6, 2023

(30) Foreign Application Priority Data
Jan. 7, 2021 (FI) 20215012

(51) Int. Cl. G01N 33/569; G01N 33/542; C12N 15/63; C07K 16/12

(52) U.S. Cl. CPC G01N 33/56938; G01N 33/542; C12N 15/63; C07K 16/1271; G01N 2333/31; C07K 2317/565; C07K 2317/622

(57) ABSTRACT

The invention relates to a method for determining a target molecule in a sample. Also, the invention relates to a Förster resonance energy transfer (FRET, fluorescence resonance energy transfer) pair, recombinant antibodies, or antigen binding fragments thereof that bind Staphylococcus aureus Enterotoxin A and a test kit comprising the FRET pair, antibody or antigen binding fragment. Furthermore, the present invention relates to an isolated nucleic acid molecule comprising a nucleotide sequence that encodes the recombinant antibody or antigen binding fragment, and an expression vector comprising said nucleic acid molecule. Still, the invention relates to use of the FRET pair or the recombinant antibody or an antigen binding fragment thereof for determining a target molecule such as Staphylococcus aureus Enterotoxin A in a sample. And still, the invention relates to a method of producing the antibody or antigen binding fragment.

### FIELD OF THE INVENTION

The present invention relates to the fields of life sciences, immunoassays and recombinant antibodies and antigen binding fragments. Specifically, the invention relates to a method for determining a target molecule in a sample. Also, the present invention relates to a Förster resonance energy transfer (FRET, fluorescence resonance energy transfer) pair, recombinant antibodies or antigen binding fragments thereof that bind Staphylococcus aureus Enterotoxin A and a test kit comprising the FRET pair, antibody or antigen binding fragment of the present invention. Furthermore, the present invention relates to an isolated nucleic acid molecule comprising a nucleotide sequence that encodes the recombinant antibody or antigen binding fragment of the present invention, and an expression vector comprising said nucleic acid molecule. Still, the present invention relates to use of the FRET pair or the recombinant antibody or an antigen binding fragment thereof of the present invention for determining a target molecule such as Staphylococcus aureus Enterotoxin A in a sample. And still, the present invention relates to a method of producing the antibody or antigen binding fragment of the present invention.

### BACKGROUND OF THE INVENTION

Competitive and non-competitive immunoassays are well known in the prior art. However, more specific and sensitive non-competitive assays are needed for detecting specific target molecules, such as those causing harm, health hazards or danger to people or animals, from any samples.

The global food supply is under constant threat of contaminations, and this gener-ates a demand for effective test products that can ensure food safety. Microbial pathogens, such as bacteria and viruses, environmental toxins, food allergens and adulterants, residues of drugs, and agricultural chemicals, are hazardous to human beings, if consumed unchecked. Pathogen testing is still the most dominant segment of food testing. Increase in applications of pesticides in agriculture and high application preservatives have been generating demand for the chemical and toxin testing market, followed by the genetically modified organism (GMO) market.

One example of harmful molecules to people and animals and in need of improved and excellent detection methods is a group of Staphylococcus aureus enterotoxins (SEs) (Zhang et al. 2019, ACS Appl. Nano Mater. 2: 4150-4158). SEs are small single domain proteins, which are stable to heating, freezing, drying and a wide range of pH (4-10) and also resistant to gut proteases (Argudin et al. 2010, Toxins 2, 1751-1773). SEs cause food poisoning and Enterotoxin A (SEA) is found to be the most frequent specific toxin involved in food poisoning caused by S. aureus (Hennekinne et al. 2012 FEMS Microbiol. Rev, 36, 815-836). More than 13000 persons in Japan were affected by SEA poisoning at year 2000 (Asao et al. 2003, Epidemiol. Infect, 130: 33). SEA concentration found was <0.4 ng/ml in low-fat milk and 3.7 ng/g in powdered skimmed milk. Indeed, for example milk serves as a good substrate for the growth of S. aureus which can contaminate raw milk but also any phase in milk processing.

For example, Wu S et al. (2013, Analytica Chimica Acta, vol. 782, 59-66) and Vi-nayaka A and Thakur M (2013, Luminescence, vol. 28, issue 6, 827-835) de-scribe FRET based bioassays for detecting staphylococcal enterotoxin B (SEB).

Still, rapid, simple, sensitive and specific in situ diagnostics are needed for moni-toring or controlling purposes of specific target molecules for example in raw mate-rials, manufactured products or in any processes of interest e.g., in food and bev-erage industries and health care. For food safety, such as prevention of food poisoning outbreaks caused by SEs, fast, simple, sensitive and specific methods and means for determining SEs, such as SEA, in biological samples are critical.

### BRIEF DESCRIPTION OF THE INVENTION

The objects of the invention, including but not limited to a method and tools for determining a target molecule in a sample, are achieved by utilizing a very specific immunoassay. Indeed, surprisingly at least two recombinant antibodies or antigen binding fragments thereof can be used for producing a Förster resonance energy transfer (FRET) reaction when binding a target molecule. A very simple, sensitive, and specific method can be obtained by the combination of antibodies or antigen binding fragments thereof and tools enabling a FRET reaction in the presence of a target antigen.

Lack of simple, rapid, non-clinical or clinical diagnostic methods and tools based on FRET can be overcome by the present invention. Furthermore, the present invention enables high throughput analysis of target molecules.

The inventors of the present disclosure were able to develop a simple and cost-effective one-step immunoassay based on FRET phenomenon for target molecules. Furthermore, two specific anti-SEA antibodies binding to different epitopes of SEA were identified and utilized in the FRET assay development.

Indeed, the method, kit, FRET pair, antibody or antigen binding fragment thereof are easy and fast to use. For example, no sample pretreatment is necessarily needed for the method or tools of the present invention. With the present invention rapid and sensitive immunoassay results can be obtained within 5-10 minutes thereby enabling effective and allocated decision making.

Advantages of the present invention include that suitable samples to be used for the present invention include any samples, e.g., solid, semi-solid, or liquid samples or samples comprising aerosols. Samples may be e.g., any environmental samples or taken from any process steps. Typically, samples from food processing in-dustry such as industrial process tanks or milk and dairy products are included within the scope of suitable samples.

Specifically, the present invention relates to a method for determining a target molecule in a sample, wherein the method comprises allowing a first recombinant antibody or antigen binding fragment thereof, which is capable of binding a target molecule (i.e. against a target molecule), and a second recombinant antibody or antigen binding fragment thereof, which is capable of binding said target molecule (i.e. against the target molecule) at one or more different binding sites compared to the first recombinant antibody or antigen binding fragment thereof, to contact with a sample, and determining the presence, absence or level of the target molecule in the sample, wherein the first recombinant antibody and the second recombinant antibody form a FRET pair when binding the target molecule, or wherein the first recombinant antibody and the second recombinant antibody are capable of generating a FRET reaction when binding the target molecule.

Also, the present invention relates to a Förster resonance energy transfer (FRET) pair comprising a first recombinant antibody or antigen binding fragment thereof that binds a target molecule and a second recombinant antibody or antigen binding fragment thereof that binds the target molecule at one or more different binding sites compared to the first recombinant antibody or antigen binding fragment thereof.

Furthermore, the present invention relates to a recombinant antibody or antigen binding fragment thereof that binds or is capable of binding Staphylococcus aureus Enterotoxin A, wherein the antibody or antigen binding fragment thereof comprises amino acids of one or more complementarity determining regions (CDRs) selected from the group comprising or consisting of a light chain region 1 comprising amino acids 24-35 of SEQ ID NO: 2, a light chain region 2 comprising amino acids 50-56 of SEQ ID NO: 2, a light chain region 3 comprising amino acids 90-98 of SEQ ID NO: 2, a heavy chain region 1 comprising amino acids 30-35 of SEQ ID NO: 1, a heavy chain region 2 comprising amino acids 50-69 of SEQ ID NO: 1, and a heavy chain region 3 comprising amino acids 99-108 of SEQ ID NO: 1.

Furthermore, the present invention relates to a recombinant antibody or antigen binding fragment thereof that binds or is capable of binding Staphylococcus aureus Enterotoxin A, wherein the antibody or antigen binding fragment thereof comprises amino acids of one or more complementarity determining regions (CDRs) selected from the group comprising or consisting of a light chain region 1 comprising amino acids 24-34 of SEQ ID NO: 4, a light chain region 2 comprising amino acids 50-56 of SEQ ID NO: 4, a light chain region 3 comprising amino acids 89-97 of SEQ ID NO: 4, a heavy chain region 1 comprising amino acids 30-35 of SEQ ID NO: 3, a heavy chain region 2 comprising amino acids 50-69 of SEQ ID NO: 3, and a heavy chain region 3 comprising amino acids 99-107 of SEQ ID NO: 3.

Furthermore, the present invention relates to a combination of at least two recombinant antibodies or antigen binding fragments of the present invention that bind or are capable of binding Staphylococcus aureus Enterotoxin A at different binding sites.

Furthermore, the present invention relates to an isolated nucleic acid molecule comprising a nucleotide sequence that encodes the recombinant antibody or antigen binding fragment of the present invention.

Still, the present invention relates to an expression vector comprising the nucleic acid molecule of the present invention.

Still, the present invention relates to a method of producing the antibody or antigen binding fragment of the present invention, wherein the method comprises introducing the nucleic acid molecule or an expression vector of the present invention into a host cell and growing the cell under conditions permitting production of the antibody or antigen binding fragment.

Still, the present invention relates to a method for determining Staphylococcus aureus Enterotoxin A in a sample, wherein the method comprises allowing a recombinant antibody or antigen binding fragment of the present invention to contact with a sample and thereafter determining the presence, absence, or level of Staphylococcus aureus Enterotoxin A in the sample.

Still furthermore, the present invention relates to a test kit comprising the FRET pair, antibody, or antigen binding fragment of the present invention.

Still, the present invention relates to use of the FRET pair, the (first or second) recombinant antibody or an antigen binding fragment thereof, or kit of the present invention for determining a target molecule such as Staphylococcus aureus enterotoxin or Staphylococcus aureus Enterotoxin A in a sample.

Still, the present invention relates to a method of (pre)treating a sample, wherein the method comprises allowing at least one antibody or an antigen binding fragment thereof of the present invention to contact with a target molecule of a sample thereby forming a complex comprising said at least one antibody or antigen binding fragment thereof and the target molecule.

Still, the present invention relates to use of the FRET pair or the (first or second) recombinant antibody or an antigen binding fragment thereof of the present inven-tion for (pre)treating a sample, which optionally comprises a target molecule such as Staphylococcus aureus enterotoxin or Staphylococcus aureus Enterotoxin A.

Other objects, details and advantages of the present invention will become appar-ent from the following drawings, detailed description, and examples.

### BRIEF DESCRIPTION OF THE DRAWINGS

FIG. 1 shows a schematic picture of the principle of the FRET immunoassay of the present invention. In one embodiment the fluorescently labelled anti-target molecule (e.g., anti-SEA) antibodies or antigen binding fragments thereof bind to the close epitopes of the target molecule (e.g., SEA) of a sample making possible the measurement of the FRET signal e.g., with a fluorometer. Thereby the pres-ence, absence, or level of the target molecule (such as Staphylococcus aureus en-terotoxin A (SEA) as shown in the FIG. 1) can be determined.

FIG. 2 shows amino acid sequences of the variable domains of heavy and light chains of the specific recombinant antibody or antigen binding fragment of the present invention (anti-SEA (55) Fab) (SEQ ID NOs: 1 and 2, respectively, CDRs are marked in bold).

FIG. 3 shows amino acid sequences of the variable domains of heavy and light chains of the specific recombinant antibody or antigen binding fragment of the present invention (anti-SEA (90) Fab) (SEQ ID NOs: 3 and 4, respectively, CDRs are marked in bold).

FIG. 4 shows polynucleotide sequences, which encode variable domains of heavy and light chains of the anti-SEA (55) Fab (SEQ ID NOs: 5 and 6, respectively).

FIG. 5 shows polynucleotide sequences, which encode variable domains of heavy and light chains of the anti-SEA (90) Fab (SEQ ID NOs: 7 and 8, respectively).

FIG. 6 shows results of the competitive ELISA for anti-SEA (55) and (90) antibod-ies. Soluble SEA in concentrations of 0-10 nM was used as a competing molecule.

FIG. 7 shows a binding site analysis of anti-SEA (55) Fab and anti-SEA (90) Fab antibodies by SPR analysis.

FIG. 8 reveals results of the FRET immunoassay for SEA in PBS-0.5% BSA buffer.

FIG. 9 reveals results of the FRET immunoassay for SEA in whole milk.

FIG. 10 reveals that the FRET immunoassay has high specificity for SEA.

FIG. 11 shows the principle of SEA luminescence complementation assay. Truncated NanoLuc® produces luminescence only when both B9 and B10 pep-tides have completed its structure, in the presence of furimazine substrate. The missing peptides B9 and B10 can be brought close enough to each other by anti-bodies binding to SEA antigen. Simultaneous binding of the two antibodies allow also simultaneous complementation of the truncated NanoLuc®, yielding a meas-urable luminescence signal, proportional to the concentration of SEA in the sam-ple.

FIG. 12 shows the amino acid sequences of the designed fusions of Example 8.

FIG. 13 shows the determination of the sensitivity and linear range of LCA for SEA fortified PBS buffer. Each concentration was measured with six replicates. Signal was measured after 5 min incubation with the substrate

FIG. 14 shows the determination of the sensitivity and linear range of LCA for SEA fortified whole, unhomogenized milk. Each concentration was measured with six replicates. Signal was measured after 5 min incubation with the substrate.

### SEQUENCE LISTING

Sequences or partial sequences of anti-target molecule antibodies or antigen binding fragments of the present invention and polynucleotide sequences encoding variable domains of heavy and light chains:

SEQ ID NO: 1: anti-SEA (55) Fab, heavy chain amino acid sequence (CDRs at amino acid positions 30-35, 50-69 and 99-108 of SEQ ID NO: 1, e.g., according to Kabat e.g. as presented in Dondelinger, M. et. al. (2018, Frontiers in Immunolo-gy, volume 9, Article 2278))

SEQ ID NO: 2: anti-SEA (55) Fab, light chain amino acid sequence (CDRs at amino acid positions 24-35, 50-56 and 90-98 of SEQ ID NO: 2, e.g., according to Kabat e.g. as presented in Dondelinger, M. et. al. (2018, Frontiers in Immunolo-gy, volume 9, Article 2278))

SEQ ID NO: 3: anti-SEA (90) Fab, heavy chain amino acid sequence (CDRs at amino acid positions 30-35, 50-69 and 99-107 of SEQ ID NO: 3, e.g., according to Kabat e.g., as presented in Dondelinger, M. et. al. (2018, Frontiers in Immunolo-gy, volume 9, Article 2278))

SEQ ID NO: 4: anti-SEA (90) Fab, light chain amino acid sequence (CDRs at amino acid positions 24-34, 50-56 and 89-97 of SEQ ID NO: 4, e.g., according to Kabat e.g., as presented in Dondelinger, M. et. al. (2018, Frontiers in Immunolo-gy, volume 9, Article 2278))

SEQ ID NO: 5 anti-SEA (55) Fab, polynucleotide sequence encoding the heavy chain of SEQ ID NO: 1

SEQ ID NO: 6 anti-SEA (55) Fab, polynucleotide sequence encoding the light chain of SEQ ID NO: 2

SEQ ID NO: 7 anti-SEA (90) Fab, polynucleotide sequence encoding the heavy chain of SEQ ID NO: 3

SEQ ID NO: 8 anti-SEA (90) Fab, polynucleotide sequence encoding the light chain of SEQ ID NO: 4.

SEQ ID NO: 9 Fab90-610-construct.

SEQ ID NO:10 B9-Fab55-construct.

### DETAILED DESCRIPTION OF THE INVENTION

The method of the present invention relates to a method for determining a target molecule in a sample, wherein the method comprises allowing a first recombinant antibody or an antigen binding fragment thereof and a second recombinant antibody or an antigen binding fragment thereof to contact with a sample, and deter-mining the presence, absence, or level of the target molecule in the sample. The first recombinant antibody and the second recombinant antibody are capable of forming a FRET pair when binding the target molecule. Indeed, the first and second recombinant antibodies or antigen binding fragments thereof are capable of generating a FRET reaction when binding the target molecule, if said target molecule is present in the sample.

A first recombinant antibody or an antigen binding fragment thereof, which can be used in the method, FRET pair or kit of the present invention, binds or is capable of binding a target molecule (i.e., is against said target molecule). A second recombinant antibody or an antigen binding fragment thereof, which can be used in the method, FRET pair or kit of the present invention, binds or is capable of binding said target molecule at one or more different binding sites compared to the first recombinant antibody or antigen binding fragment thereof. The second antibody or antigen binding fragment is thus against the same target molecule as the first antibody or antigen binding fragment.

The present invention also relates to a specific FRET pair comprising two different (named first and second) recombinant antibodies or antigen binding fragments thereof against a target molecule, wherein each antibody or antigen binding fragment binds said target molecule at a different binding site compared to another antibody or antigen binding fragment (or other antibodies or antigen binding frag-ments). Indeed, the FRET pair may comprise two recombinant antibodies, two antigen binding fragments, or one recombinant antibody and one antigen binding fragment. Also, specific recombinant antibodies or antigen binding fragments thereof are within the scope of the present invention.

In FRET, energy from a molecular donor (a fluorophore) is excited to a high-energy state and transferred to an acceptor (e.g., another fluorophore) via intermo-lecular coupling. The donor is always a fluorescent molecule. Transfer of energy from the donor to the acceptor is possible only if the distance between the donor and the acceptor is short (typically e.g., 10-100 Å) and the fluorescence emission spectrum of the donor and the absorption or excitation spectrum of the acceptor partially overlap. The energy transfer is then detected as a change in fluores-cence. Often time-resolved fluorescence is utilized (e.g., Hemmila I et al., 1988, Clin. Chem. 34: 2320-2322). In general, the donor and acceptor moieties are different, in which case FRET can be detected by the appearance of fluorescence of the acceptor or by quenching of donor fluorescence. For FRET e.g., review of Algar, W.R. et al (2019, Nature methods 16:815-829) can be consulted.

FRET can be applied to the method for determining a target molecule by labelling at least two selected molecules from the group consisting of i) a first recombinant antibody or an antigen binding fragment thereof, and ii) a second recombinant antibody or an antigen binding fragment thereof. Any recombinant antibody or antigen binding fragment of the present invention can be labelled. In one embodiment labels are fluorophores that form a FRET donor-acceptor pair. Fluorophores in-clude but are not limited to small organic dyes (such as fluorescent dyes), fluores-cent proteins (FPs), quantum dots (QDs), core-shell nanoparticles and time-resolved nanoparticles. For example, one or more following donor-acceptor pairs are suitable for the present invention: FITC (e.g., emission 520 nm)-TRITC (e.g., excitation 550 nm), Cy3 (e.g., emission 566 nm)-Cy5 (e.g., excitation 649 nm), EGFP (e.g., emission 508 nm)-Cy3 (e.g., excitation 554 nm), CFP (e.g. emission 477 nm)—YFP (e.g. excitation 514 nm), EGFP (e.g. emission 508 nm)—YFP (e.g. excitation 514 nm). Other suitable FRET donor-acceptor pairs include but are not limited to Europium chelate—Alexa (e.g., 647), Cy3.5.—graphene oxide and Ianthanide—QD. A measurable FRET signal can be obtained when the labels such as fluorophores come into very close proximity.

Labeling of at least one, two or more antibodies or antigen binding fragments can be either direct labeling or indirect labeling e.g., through any molecule or particle. In one embodiment the first antibody or antigen binding fragment thereof and/or the second antibody or antigen binding fragment thereof comprises a label; either the first or second antibody or antigen binding fragment thereof comprises an acceptor label, optionally an acceptor fluorophore label, and/or the other of the first and second antibodies or antigen binding fragments thereof comprises a donor label, optionally a donor fluorophore label; and/or the first antibody or antigen binding fragment thereof and the second antibody or antigen binding fragment thereof are a pair for a sandwich (e.g. ELISA) assay. In one embodiment one, two or more recombinant antibodies or antigen binding fragments are fluorescently labelled recombinant antibodies.

In a very specific embodiment, an antibody or antigen binding fragment such as anti-SEA (90) Fab has been labelled with a donor fluorophore (e.g., Europium chelate) and another antibody or antigen binding fragment such as anti-SEA (55) Fab has been labelled with an acceptor fluorophore (e.g., Alexa 647), respectively.

The obtained FRET reaction can be measured or detected by any suitable method known to a person skilled in the art. Suitable FRET measurement or detection methods include but are not limited to an acceptor photobleaching, donor photo-bleaching, ratio imaging, sensitized emission and fluorescence lifetime measure-ments. In one embodiment FRET signals are detected by TR-fluorometer (such as TR-fluorometer, e.g., Victor, PerkinElmer) or FRET reader (for example after 1-60 min, 1-50 min, 1-40 min, 1-30 min, 1-20 min, 1-10 min or 5-10 min in-cubation or mixing of the sample and the antibodies or antigen binding fragments). For example, multiplexed photonic sensor or immunosensor or any reusable immunosensor can be used for (plasmonic-based online) detection of contaminants in a sample. Among other assays, e.g., ELISA, fusion proteins-based assays and immunobead-based assays are suitable for determining target molecules by utilizing FRET phenomenon.

In one embodiment of the present invention the method comprises addition of a sample to a container (such as a well, tube or vial), chip or plate comprising at least the two antibodies of antigen binding fragments (i.e. allowing a first recombinant antibody or antigen binding fragment thereof and a second recombinant antibody or antigen binding fragment thereof to contact with a sample), and measuring or detecting FRET (i.e. thereby determining the presence, absence or level of the target molecule in the sample). Further method steps are not necessarily needed. Indeed, in one embodiment the method is a one-step immunoassay method. As used herein “one-step immunoassay method” refers to an assay, wherein only one step, i.e. contacting at least two recombinant antibodies or antigen binding fragments thereof with a sample, is needed before detection or measurements. In one embodiment at least two recombinant antibodies or antigen binding fragments thereof are allowed to contact with a sample, a combination of at least the antibodies or antigen binding fragments and the sample is mixed, and the presence, absence or level of the target molecule in the sample is determined. The antibodies or antigen binding fragments and the sample can be allowed to contact e.g., in a container (such as a well, tube or vial), chip or plate, and at the time of contacting or thereafter be mixed.

The method of the present invention is simple and fast. For example, the method for determining a target molecule can take only about 1-60 minutes, 1-50 minutes, 1-40 minutes, 1-30 minutes, 1-20 minutes or 1-15 minutes, e.g., about 5-10 minutes.

In one embodiment the first and second antibodies or antigen binding fragments thereof are allowed to contact with the sample simultaneously or sequentially (e.g., the first antibody or an antigen binding fragment thereof is allowed to contact with the sample before the second antibody or an antigen binding fragment thereof; or the second antibody or an antigen binding fragment thereof is allowed to contact with the sample before the first antibody or an antigen binding fragment thereof).

Optionally control samples (e.g., positive or negative control samples), a quality control and/or reference levels revealing specific target molecule levels or concentrations may also be included in the method of the present invention. Optionally determining a target molecule may also comprise use of any suitable statistical methods known to a person skilled in the art.

In one embodiment the method, FRET pair, recombinant antibody or antigen binding fragment or kit of the present invention enables a homogenous non-competitive immunoassay, i.e. an immunoassay that is carried out in a solution. As used herein “a non-competitive immunoassay” refers to e.g., an immunocomplex assay (Pulli, Hoyhtya, Soderlund, Takkinen, 2005, Anal. Chem. 77, 2637-2642). In noncompetitive immunoassays the intensity of the signal is directly proportional to the amount of the analyte in the sample. For example, the avoidance of immobilizing and washing steps makes the method or assay extremely simple. The present invention allows a simple one-step immunoassay for determining one or more target molecules in a sample. In one embodiment the method or assay comprises determining or detecting one, two, three, four, five or more target molecules simultaneously, or the FRET pair or kit is for determining or detecting one, two, three, four, five or more target molecules simultaneously.

The present invention provides a convenient and rapid analytical method and tools for target molecules. For example, specific target proteins and polypeptides can be used as biomarkers of health status or disease, specific therapeutic proteins or polypeptides can be monitored from samples, specific protein production of bio-processes can be monitored, and quality controls of samples can be carried out by determining specific proteins or polypeptides indicating e.g., hygiene or activity of interest. Food safety testing refers to the inspection of food products e.g., for toxins or disease-causing organisms via toxin detection.

A common feature of the target molecules or analytes suitable to be determined by the present invention is that they are typically big enough for two antibodies or fragments thereof recognizing different epitopes of said antigen, and thus typically suitable e.g., for conventional sandwich assays. The molecular mass of these ana-lytes or target molecules is typically more than about 1 kDa. In one embodiment of the invention the target molecule to be determined by the method or kit, or bound by the FRET pair, antibody or antigen binding fragment of the invention, has a molecular mass about 1-1000 kDa, about 5-1000 kDa, about 10-1000 kDa, about 1-500 kDa, about 5-500 kDa, about 10-500 kDa, about 1-100 kDa, about 5-100 kDa, about 10-100 kDa, about 1-50 kDa, about 5-50 kDa, about 10-50 kDa, about 1-30 kDa, about 5-30 kDa, about 10-30 kDa, about 20-30 kDa, or about 25-30 kDa. In one embodiment of the invention the target molecule to be determined by the method or kit, or bound by the FRET pair, antibody or antigen binding fragment of the invention, has a molecular mass at least about 5 kDa, 5.5 kDa, 6 kDa, 6.5 kDa, 7 kDa, 7.5 kDa, 8 kDa, 8.5 kDa, 9 kDa, 9.5 kDa, or 10 kDa. In one embodiment the target molecule is a protein or poly-peptide, which is optionally soluble and/or in a complex with other molecules such as proteins or polypeptides. In one embodiment the target molecule or analyte to be determined by the present invention is a monomer or a single polypeptide chain, i.e., for example not a molecule or protein comprising two or more polypeptide chains.

The target molecule can be e.g., one or more Staphylococcus aureus enterotoxins, e.g., Staphylococcus aureus Enterotoxin A (SEA) or a Staphylococcus aureus enterotoxin having at least 50%, 55%, 60%, 65%, 70%, 75%, 80%, 85%, 90% or 95% amino acid sequence identity with SEA (such as SED or SEE). Indeed, one object of the present invention has been development of a method or recombinant antibodies (rAbs) for the detection of Staphylococcus aureus enterotoxins including but not limited to enterotoxins A and/or Staphylococcus aureus enterotoxins having at least 50%, 55%, 60%, 65%, 70%, 75%, 80%, 85%, 90% or 95% amino acid sequence identity with SEA (such as SED or SEE). Staphylococcus aureus enterotoxins (SEs) belong to the broad family of pyrogenic toxin superantigens (SAgs) and are small single domain proteins stable to heating, freezing, drying and a wide range of pH (4-10). They are also resistant to gut proteases. Staphylococcal enterotoxins have been proposed to be named according to their emetic activities (Lina, G et al. J. Infect. Dis. 2004, 189, 2334-2336). Only those SAgs that induce vomiting after oral administration in a primate model are designated as SEs. Related toxins that lack emetic activity or have not been tested for it are designated as staphylococcal enterotoxin-like (SE/s) SAgs. Also, newly discovered toxins e.g., with more than 90% amino acid sequence identity with existing SEs or SE/s can be designated as a subtype. Staphylococcus aureus enterotoxins include but are not limited to (i) the classical SEA, SEB, SEC and variants thereof (such as the SEC1, SEC2 and SEC3, SEC ovine and SEC bovine variants), SED and SEE, and any variants thereof; and (ii) the new types of SEs (e.g. SEG, SEH, SEI, SER, SES, SET) and SE/s (e.g. SE/J, SE/K, SE/L, SE/M, SE/N, SE/0, SE/P, SE/Q, SE/U, SE/U2, and SE/V). In one embodiment maximum residue limits (MRLs) for SEA e.g., in milk or buffer are <1 ng/ml. As used herein expression “maximum residue limits” refers to the highest levels of a residue (i.e. target molecule) that is legally tolerated in or on food or feed. Milk is a rich substrate for the growth of S. aureus potentially producing enterotoxins of which SEA is the most frequent toxin involved in food poisoning. In one embodiment of the invention S. aureus enterotoxin is enterotoxin A. In one embodiment, S. aureus enterotoxin is enterotoxin A or a Staphylococcus aureus enterotoxin having at least 50%, 55%, 60%, 65%, 70%, 75%, 80%, 85%, 90% or 95% amino acid sequence identity with SEA (such as SED or SEE).

In one embodiment the method or tools of the present invention can be used for determining any levels or concentration of the target molecules, including but not limited to the levels below and above the maximum residue limit requirements, such as less than 1 ng/ml in a sample (e.g., buffer or milk). For example, the absence, a decreased level or a low level of the target molecules of interest can indicate a positive or good situation; and/or the presence, an increased level or a high level of the target molecules of interest can indicate a negative or poor situation; or vice versa. Indeed, the presence, absence, level, increase or decrease of the target molecule in a sample detected by the present invention (method, FRET pair, antibody, antigen binding fragment, or kit) can be used for making further decisions e.g. concerning products or processes.

Antibodies or antigen binding fragments having affinity or high affinity or improved affinity for the target molecule (such as a S. aureus enterotoxin or S. aureus enterotoxin A) can be used in the present invention or can be obtained by the present invention.

As used herein “an antibody or antigen binding fragment” refers to any antibody or a fragment thereof which is capable of binding a target molecule. The term includes e.g. any fragments or single chain antibodies (e.g. Fab, scFv) having the desired biological activities. As an example, any complementarity determining regions, heavy chain variable regions, light chain variable regions and any combinations thereof are included within the scope of “antigen binding fragments”. As used in this context “a fragment” refers to any part of a polypeptide or protein.

As used herein “antibodies” refer to polypeptides or proteins produced by immune cells or by recombinant techniques e.g. in response to antigens. An antibody is an immunoglobulin molecule and it can belong to any of classes IgG, IgM, IgE, IgA or IgD; IgG and IgM being the most frequently used. As used herein “binding poly-peptides” refer to polypeptides which bind to antigens of interest. In some embodiments binding polypeptides may inhibit or suppress the function of antigens (e.g. polypeptides or proteins) of interest.

In antibodies variable loops between n-strands, three on each light (V_{L}) and heavy (V_{H}) chain, are responsible for binding to the antigen. These loops are referred to as the complementarity determining regions (CDRs). As used herein, the term “hypervariable region” refers to the amino acid residues of an antibody or fragment thereof which are responsible for antigen binding. The hypervariable region comprises amino acid residues from a CDR. “Framework Region” or “FR” residues are those variable domain residues other than the very specific hypervariable region residues defined by residue numbers in this disclosure. As used herein, the terms “heavy chain variable domain,” “V_{H} domain” and/or “V_{H}” are used interchangeably and reference the variable domain (encompassing both the CDR and framework regions) of the heavy chain of an antibody; the terms “light chain variable domain,” “V_{L} domain” and/or “V_{L}” are used interchangeably and reference the variable do-main (encompassing both the CDR and framework regions) of the light chain of an antibody.

In one embodiment of the present invention the antibody or antigen binding fragment is a fusion polypeptide. The antibody or antigen binding fragment may have been fused (e.g., by genetic engineering) with e.g., any other fragment(s) or poly-peptide(s) to form a fusion polypeptide. Examples of suitable fusion partners in-clude any conventional fusion partners known to a person skilled in the art, e.g., including but not limited to one or more enzymes, e.g., alkaline phosphatase, hy-drophobin, any (poly)peptide suitable for functionalization, immobilization tags, and/or labelling tags (e.g., poly-Lys, fluorescent proteins). Furthermore, suitable fusion partners include any partners chemically conjugated to the antibody or antigen binding fragment in question.

At the molecular level, an antigen, ligand or target molecule can be characterized by its ability to bind to an antibody's variable Fab region or antigen binding fragment. As used herein “an antigen”, “a ligand” or “a target molecule” is a molecule that forms a complex with an antibody or a fragment thereof, e.g., with an antibody Fab or scFv.

In one embodiment of the invention, the (first) antibody or antigen binding fragment comprises amino acids of one or more (e.g. at least two, at least three, at least four, at least five or at least six) complementarity determining regions (CDRs) selected from the group comprising or consisting of a light chain region 1 comprising amino acids 24-35 of SEQ ID NO: 2, a light chain region 2 comprising amino acids 50-56 of SEQ ID NO: 2, a light chain region 3 comprising amino acids 90-98 of SEQ ID NO: 2, a heavy chain region 1 comprising amino acids 30-35 of SEQ ID NO: 1, a heavy chain region 2 comprising amino acids 50-69 of SEQ ID NO: 1, and a heavy chain region 3 comprising amino acids 99-108 of SEQ ID NO: 1, and any combination thereof.

In one embodiment of the invention, the (second) antibody or antigen binding fragment comprises amino acids of one or more (e.g. at least two, at least three, at least four, at least five or at least six) complementarity determining regions (CDRs) selected from the group comprising or consisting of a light chain region 1 comprising amino acids 24-34 of SEQ ID NO: 4, a light chain region 2 comprising amino acids 50-56 of SEQ ID NO: 4, a light chain region 3 comprising amino acids 89-97 of SEQ ID NO: 4, a heavy chain region 1 comprising amino acids 30-35 of SEQ ID NO: 3, a heavy chain region 2 comprising amino acids 50-69 of SEQ ID NO: 3, and a heavy chain region 3 comprising amino acids 99-107 of SEQ ID NO: 3, and any combination thereof.

The present invention also concerns a (first) recombinant antibody or antigen binding fragment thereof that binds Staphylococcus aureus Enterotoxin A, wherein the antibody or antigen binding fragment thereof comprises amino acids of one or more (e.g. at least two, at least three, at least four, at least five or at least six) complementarity determining regions (CDRs) selected from the group comprising or consisting of a light chain region 1 comprising amino acids 24-35 of SEQ ID NO: 2, a light chain region 2 comprising amino acids 50-56 of SEQ ID NO: 2, a light chain region 3 comprising amino acids 90-98 of SEQ ID NO: 2, a heavy chain region 1 comprising amino acids 30-35 of SEQ ID NO: 1, a heavy chain region 2 comprising amino acids 50-69 of SEQ ID NO: 1, and a heavy chain region 3 comprising amino acids 99-108 of SEQ ID NO: 1, and any combination thereof.

In one embodiment of the invention (method, antibody or fragment, FRET pair, or kit), the (first) antibody or antigen binding fragment comprises the light chain CDR3 as set forth in amino acids 90-98 of SEQ ID NO:2, a CDR2 as set forth in amino acids 50-56 of SEQ ID NO:2 and/or a CDR1 as set forth in amino acids 24-35 of SEQ ID NO:2. In one embodiment of the invention the recombinant antibody or antigen binding fragment comprises the heavy chain CDR3 as set forth in amino acids 99-108 of SEQ ID NO:1, a CDR2 as set forth in amino acids 50-69 of SEQ ID NO:1 and/or a CDR1 as set forth in amino acids 30-35 of SEQ ID NO:1.

In one embodiment the recombinant antibody or antigen binding fragment comprises the light chain CDR3 as set forth in amino acids 90-98 of SEQ ID NO:2, and optionally either the CDR2 as set forth in amino acids 50-56 of SEQ ID NO:2 or CDR1 as set forth in amino acids 24-35 of SEQ ID NO:2; and/or the heavy chain CDR3 as set forth in amino acids 99-108 of SEQ ID NO:1, and optionally either the CDR2 as set forth in amino acids 50-69 of SEQ ID NO:1 or CDR1 as set forth in amino acids 30-35 of SEQ ID NO:1.

In one embodiment of the invention the recombinant antibody or antigen binding fragment comprises the heavy chain CDR1 as set forth in amino acids 30-35 of SEQ ID NO:1, the CDR2 as set forth in amino acids 50-69 of SEQ ID NO:1, and the CDR3 as set forth in amino acids 99-108 of SEQ ID NO:1; and the light chain CDR3 as set forth in amino acids 90-98 of SEQ ID NO:2.

In one embodiment of the invention the recombinant antibody or antigen binding fragment comprises the heavy chain CDR1 as set forth in amino acids 30-35 of SEQ ID NO:1, the CDR2 as set forth in amino acids 50-69 of SEQ ID NO:1, and the CDR3 as set forth in amino acids 99-108 of SEQ ID NO:1; and the light chain CDR3 as set forth in amino acids 90-98 of SEQ ID NO:2, and either the CDR1 as set forth in amino acids 24-35 or the CDR2 as set forth in amino acids 50-56 of SEQ ID NO:2.

In one embodiment (of the method, antibody or fragment, FRET pair, or kit), the (first) antibody or antigen binding fragment thereof comprises an amino acid se-quence having 80-100% sequence identity, e.g. at least 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% or 100% sequence identity to SEQ ID NO: 1 (heavy chain amino acid sequence, (55)) and/or to SEQ ID NO: 2 (light chain amino acid sequence, (55)), e.g. having at least 90% sequence identity to SEQ ID NO: 1 (heavy chain amino acid sequence, (55)) and/or SEQ ID NO: 2 (light chain amino acid sequence, (55)); or the heavy chain of the (first) antibody or an antigen binding fragment thereof comprises SEQ ID NO: 1, and/or the light chain of the (first) antibody or an antigen binding fragment thereof comprises SEQ ID NO: 2. In one embodiment, the heavy chain variable region (V_{H}) of the recombinant antibody or antigen binding fragment comprises or consists of amino acids of SEQ ID NO: 1 and/or the light chain variable region (V_{L}) of the recom-nant antibody or antigen binding fragment comprises or consists of amino acids 24-98 of SEQ ID NO: 2.

The present invention further concerns a (second) recombinant antibody or antigen binding fragment thereof that binds Staphylococcus aureus Enterotoxin A, wherein the antibody or antigen binding fragment thereof comprises amino acids of one or more (e.g. at least two, at least three, at least four, at least five or at least six) complementarity determining regions (CDRs) selected from the group comprising or consisting of a light chain region 1 comprising amino acids 24-34 of SEQ ID NO: 4, a light chain region 2 comprising amino acids 50-56 of SEQ ID NO: 4, a light chain region 3 comprising amino acids 89-97 of SEQ ID NO: 4, a heavy chain region 1 comprising amino acids 30-35 of SEQ ID NO: 3, a heavy chain region 2 comprising amino acids 50-69 of SEQ ID NO: 3, and a heavy chain region 3 comprising amino acids 99-107 of SEQ ID NO: 3, and any combination thereof.

In one embodiment of the invention (method, antibody or fragment, FRET pair, or kit), the (second) antibody or antigen binding fragment comprises the light chain CDR3 as set forth in amino acids 89-97 of SEQ ID NO:4, the CDR2 as set forth in amino acids 50-56 of SEQ ID NO:4, and/or the CDR1 as set forth in amino acids 24-34 of SEQ ID NO:4. In one embodiment of the invention the recombinant antibody or antigen binding fragment comprises the heavy chain CDR3 as set forth in amino acids 99-107 of SEQ ID NO:3, the CDR2 as set forth in amino acids 50-69 of SEQ ID NO:3, and/or the CDR1 as set forth in amino acids 30-35 of SEQ ID NO:3.

In one embodiment the recombinant antibody or antigen binding fragment comprises the light chain CDR3 as set forth in amino acids 89-97 of SEQ ID NO:4, and optionally either the CDR2 as set forth in amino acids 50-56 of SEQ ID NO:4 or the CDR1 as set forth in amino acids 24-34 of SEQ ID NO:4; and/or the heavy chain CDR3 as set forth in amino acids 99-107 of SEQ ID NO:3, and optionally ei-ther the CDR2 as set forth in amino acids 50-69 of SEQ ID NO:3 or the CDR1 as set forth in amino acids 30-35 of SEQ ID NO:3.

In one embodiment of the invention the recombinant antibody or antigen binding fragment comprises the heavy chain CDR1 as set forth in amino acids 30-35 of SEQ ID NO:3, the CDR2 as set forth in amino acids 50-69 of SEQ ID NO:3, and the CDR3 as set forth in amino acids 99-107 of SEQ ID NO:3; and the light chain CDR3 as set forth in amino acids 89-97 of SEQ ID NO:4.

In one embodiment of the invention the recombinant antibody or antigen binding fragment comprises the heavy chain CDR1 as set forth in amino acids 30-35 of SEQ ID NO:3, the CDR2 as set forth in amino acids 50-69 of SEQ ID NO:3, and the CDR3 as set forth in amino acids 99-107 of SEQ ID NO:3; and the light chain CDR3 as set forth in amino acids 89-97 of SEQ ID NO:4, and either the CDR1 as set forth in amino acids 24-34 or the CDR2 as set forth in amino acids 50-56 of SEQ ID NO:4.

In one embodiment (of the method, antibody or fragment, FRET pair, or kit), the (second) antibody or antigen binding fragment thereof comprises an amino acid sequence having 80-100% sequence identity, e.g. at least 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% or 100% sequence identity to SEQ ID NO: 3 (heavy chain amino acid sequence, (90)) and/or to SEQ ID NO: 4 (light chain amino acid sequence, (90)), e.g. having at least 90% sequence identi-ty to SEQ ID NO: 3 (heavy chain amino acid sequence, (90)) and/or SEQ ID NO: 4 (light chain amino acid sequence, (90)); or the heavy chain of the second antibody or an antigen binding fragment thereof comprises SEQ ID NO: 3, and/or the light chain of the second antibody or an antigen binding fragment thereof comprises SEQ ID NO: 4. In one embodiment, the heavy chain variable region (V_{H}) of the recombinant antibody or antigen binding fragment comprises or consists of amino acids 30-107 of SEQ ID NO: 3 and/or the light chain variable region (V_{L}) of the recombinant antibody or antigen binding fragment comprises or consists of amino acids 24-97 of SEQ ID NO: 4.

In a further embodiment of the invention an isolated nucleic acid molecule encoding the antibody or antigen binding fragment of the present invention comprises a polynucleotide sequence having 50-100%, 60-100%, 70-100% or 80-100% sequence identity, e.g. at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 5, 6, 7 and/or 8 (e.g. 5 and 6; or 7 and 8), e.g. having at least 90% sequence identity to SEQ ID NO: 5, 6, 7 and/or 8. In one embodiment the polynucleotide encodes the heavy chain variable region (V_{H}) of the recombinant antibody or antigen binding fragment comprising or consisting of amino acids 30-108 of SEQ ID NO: 1 and/or the light chain variable region (V_{L}) of the recombinant antibody or antigen binding fragment comprising or consisting of amino acids 24-98 of SEQ ID NO: 2. In one embodiment the polynucleotide encodes the heavy chain variable region (V_{H}) of the recombinant antibody or antigen binding fragment comprising or consisting of amino acids 30-107 of SEQ ID NO: 3 and/or the light chain variable region (V_{L}) of the recombinant antibody or antigen binding fragment comprising or consisting of amino acids 24-97 of SEQ ID NO: 4.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e. identity=# of identical positions/total # of positions×100), taking into account the number of gaps, and the length of each gap, which need to be introduced for opti-mal alignment of the two sequences. The comparison of sequences and determi-nation of identity percentage between two sequences can be accomplished using mathematical algorithms available in the art. This applies to both amino acid and nucleic acid sequences. As an example, sequence identity may be determined by using BLAST (Basic Local Alignment Search Tools) or FASTA (FAST-All). In the searches, setting parameters “gap penalties” and “matrix” are typically selected as default. In one embodiment the sequence identity is determined against the full length sequence of the present disclosure.

In one embodiment recombinant antibodies or fragments (such as anti-SEA (55) Fab and anti-SEA (90) Fab) e.g. used in the method, FRET pair or kit of the present invention, have unique amino acid and nucleotide sequences. In one embod-iment both antibodies have high affinity for one or more Staphylococcus aureus enterotoxins such as SEA.

In one embodiment of the invention the antigen binding fragment is a single chain Fv (scFv) or Fab fragment. In one embodiment of the method or FRET pair of the present invention, the antigen binding fragment of the first and/or second recombi-nant antibody is a single chain Fv (scFv) or Fab fragment. “Fv” (variable domain) refers to the variable regions of the immunoglobulin molecule that are responsible for the ligand binding. “A single-chain Fv fragment (scFv)” refers to a fragment comprising the V_{H} and the V_{L} domains (variable regions) of the immunoglobulin molecule connected by a linker in a single polypeptide synthesized from a single mRNA molecule. As used herein “a Fab fragment” (fragment antigen-binding) re-fers to a region of an antibody consisting of the variable and constant domains of an immunoglobulin light chain covalently attached by a disulfide bridge to the vari-able and first constant domain of an immunoglobulin heavy chain.

In one embodiment of the method, FRET pair, antibody or a fragment thereof, or kit of the present invention, the antibody, e.g. the first or second antibody or antigen binding fragment thereof (such as anti-SEA (55) Fab) has an affinity of 50 nM or less, e.g. 1.0-50 nM, 2.0-50 nM, 2.0-40 nM, 2.0-30 nM, 2.0-20 nM, 2.0-10 nM, 2.0-6.0 nM, 2.5-5 nM, 3.0-4.5 nM or 3.5-4.0 nM towards Staphylococcus aureus enterotoxin or Staphylococcus aureus Enterotoxin A; and/or the first or second antibody or antigen binding fragment thereof (such as anti-SEA (90) Fab) has an affinity of 50 nM or less, e.g. 1.0-50 nM, 1.5-50 nM, 1.5-40 nM, 1.5-30 nM, 1.5-20 nM, 1.5-10 nM, 1.5-6.0 nM, 2.0-5 nM, 2.5-4.5 nM, 2.5-4.0 nM or 2.5-3.5 nM towards Staphylococcus aureus enterotoxin or Staphylococcus aureus Enterotoxin A (e.g. when measured by Surface Plasmon Reso-nance (SPR) sensors and/or method (e.g. Biacore T200), e.g. as described in ex-ample 5 of the present disclosure). In a very specific embodiment the first antibody or antigen binding fragment thereof (such as anti-SEA (55) Fab) has an affinity of nM or less, e.g. 1.0-50 nM, 2.0-50 nM, 2.0-40 nM, 2.0-30 nM, 2.0-20 nM, 2.0-10 nM, 2.0-6.0 nM, 2.5-5 nM, 3.0-4.5 nM or 3.5-4.0 nM, and the second antibody or antigen binding fragment thereof (such as anti-SEA (90) Fab) has an affinity of 50 nM or less, e.g. 1.0-50 nM, 1.5-50 nM, 1.5-40 nM, 1.5-30 nM, 1.5-20 nM, 1.5-10 nM, 1.5-6.0 nM, 2.0-5 nM, 2.5-4.5 nM, 2.5-4.0 nM or 2.5-3.5 nM towards Staphylococcus aureus enterotoxin or Staphylococcus aureus Enterotoxin A.

Indeed, advantages of the present invention include but are not limited to good or high affinity and specificity.

As used herein “specificity” refers to a degree revealing how the recombinant antibody or antigen binding fragment of the present invention or used in the present invention is able to detect a target molecule of interest in relation to the other tar-get molecules.

In one embodiment the recombinant antibody or antigen binding fragment of the invention against Staphylococcus aureus Enterotoxin A has only some cross reac-tivity or no cross reactivity to Staphylococcus aureus Enterotoxin B. In a very spe-cific embodiment of the invention the cross reactivity is measured by utilizing the SPR method and/or (SPR) sensors (e.g., Biacore T200) (e.g., as described in ex-ample 5 of the present disclosure). As used herein “cross reactivity” refers to a re-action between an antibody or antigen binding fragment and an antigen other than the most specific antigen to said antibody or antigen binding fragment. Indeed, cross-reactivity measures the extent to which different antigens appear similar to an antibody or an antigen binding fragment.

The present invention further concerns a method of producing a recombinant antibody or antigen binding fragment of the present invention. The method comprises introducing the nucleic acid molecule or an expression vector of the present inven-tion into a host cell and growing the cell under (suitable) conditions permitting pro-duction of the antibody or antigen binding fragment, and optionally further recovering the produced antibody or antigen binding fragment. As used herein, “(suitable) conditions permitting production of the antibody or antigen binding fragment” mean any conditions allowing survival or growth of a host cell, and/or production of the desired antibody or antigen binding fragment in the host cell.

The present invention also concerns an expression vector comprising the nucleic acid molecule of the present invention encoding the recombinant antibody or antigen binding fragment of the present invention.

Mammalian cell lines are widely used expression systems for the production of recombinant antibodies. Bacteria, yeasts, filamentous fungi, plant and insect cells can also be employed for the production of recombinant antibodies in order to lower the production costs in cases wherein the glycosylation pattern does not play a critical role (e.g. in vitro diagnostics). Therefore, a host cell comprising the recombinant nucleic acid molecule encoding the antibody or antigen binding fragment or the expression vector comprising the recombinant nucleic acid molecule is not limited to but may be selected e.g., from the group consisting of eukaryotic or prokaryotic cell, bacteria, yeasts, filamentous fungi, and animal, human, mammalian, plant and insect cell lines. It may even be a hybridoma cell, which after trans-formation produces a recombinant monoclonal antibody.

Recombinant antibodies or antigen binding fragments can be developed e.g., by using genetically engineered genes expressed in an in vitro cell line or by tradi-tional hybridoma based technologies. The recombinant antibody or antigen binding fragment may also be produced synthetically. All these techniques for producing recombinant antibodies are known to a person skilled in the art and described in practical manuals and handbooks describing laboratory molecular techniques. Recombinant techniques in producing antibodies or antigen binding fragments enable production of identical antibodies in each manufacturing batch. Furthermore, the manufacturing process gives high purity and ultra-low batch-to-batch variability.

As used herein “a recombinant antibody or antigen binding fragment” refers to an antibody or antigen binding fragment (e.g., Fab and/or scFv), which has been obtained by genetically modifying genetic material or has been produced by a recombinant DNA technology. Therefore, a recombinant antibody or antigen binding fragment can comprise mutations compared to the (corresponding) wild type antibody or antigen binding fragment (e.g., comprise a deletion, substitution, disrup-tion or insertion of one or more amino acids). In one embodiment “a recombinant antibody or antigen binding fragment” comprises or is a polypeptide encoded by a polynucleotide that has been cloned in a system that supports expression of said polynucleotide and furthermore translation of said polypeptide. In one embodiment “recombinant antibodies” further include recombinant or synthetic monoclonal antibodies, e.g., produced from a specific Fab (such as anti-SEA (50) or (90) Fab) optionally by utilizing reverse engineering.

As is known by a person skilled in the art, for example antigen binding fragment libraries such as antibody phage display libraries can be used for discovery and development of recombinant antibodies or antigen binding fragments thereof. The recombinant antibody or antigen binding fragment library is conveniently an expression library, which is typically a phage display library. The general principle of the display recombinant antibody or antigen binding fragment libraries is that they present the antigen binding fragment as a fusion protein on the surface, which may be the surface of a microbial cell such as a yeast or bacterial cell, or a bacteriophage. The display recombinant library can also be a display library, where stable complexes of nascent protein and mRNA are produced in an in vitro expression system. Phage display is the most frequently used display method for antibody libraries. The antibodies or antigen binding fragments are conveniently discovered from a recombinant phage display antibody library.

A phage display antibody library may be constructed by antibody fragment coding DNAs into an appropriate phage display vector. DNA encoding for millions of variants of antibody fragments is batch-cloned into the vector as part of the phage coat protein. Large libraries containing millions of antibody fragments with different specificities can be obtained by transforming the vectors to bacteria. Cultivation of the bacteria leads to the expression of phages displaying antibody fragments on the surface of the phages. The gene for the displayed antibody can be carried in the phagemid plasmid packed inside the phage, thus linking genotype with pheno-type. The physical linkage between the displayed protein and its DNA allows screening of vast numbers of variants of the protein, each linked to its corresponding DNA, by a simple in vitro selection procedure. Optionally, the in vitro selection comprises incubating the pool of phage displayed variants with the antigen or ligand of interest that has been immobilized on a carrier, washing away unbound phage, and eluting specifically bound phage by disrupting the binding to the ligand. The eluted phage can then be amplified in vivo. For example, after several rounds of selection followed by a screening assay for individual antibody clones, the best clones can be sequenced and the diversity of the binders can be examined.

Subsets of strains containing antibodies or antigen binding fragments of the present invention (anti-SEA (55) Fab, anti-SEA (90) Fab) have been deposited according to the Budapest Treaty on the International Recognition of the Deposit of Microorganisms for the Purposes of Patent Procedure, in VTT Culture Collection, P.O. Box 1000, FI-02044 VTT, FINLAND (http://culturecollection.vtt.fi) on Nov. 12th, 2020, with the following accession numbers, respectively: VTT E-203593 and VTT E-203594.

The present invention further concerns a test kit comprising the FRET pair, antibody or antigen binding fragment of the present invention. Also, the present inven-tion concerns said test kit for use in a method of the present invention or for determining a target molecule of interest. In one embodiment the present invention relates to a fast and simple kit for various diagnostics platforms and sensors for determining a target molecule in a sample. In one embodiment the test kit may further comprise one or more reagents for performing a test or a method of the present invention, and optionally instructions for use or performing a test or method. In one embodiment the test kit comprises one or several reagents selected from the group consisting of reaction solutions, buffers, washing solutions and detection means, such as labels and a fluorometer.

In one embodiment said test kit is an immunoassay kit, and in a further embodiment said immunoassay is a competitive or non-competitive immunoassay. For example, when the kit comprises a FRET pair or a combination of the antibodies or antigen binding fragments of the invention (e.g., two Fabs), the kit can be a non-competitive immunoassay kit. And for example, when the kit comprises at least one antibody or antigen binding fragment of the invention, the kit can be a competitive or non-competitive immunoassay kit, e.g., a conventional ELISA test or a lateral flow-test. Sensor assays for polypeptide antigens can be direct assays, competitive assays or sandwich assays. Direct and competitive assays use one antibody for the antigen while sandwich assay utilizes two antibodies binding to the different epitopes of the antigen. Indeed, one or more recombinant antibodies or antigen binding fragments of the present invention can be used in any kind of test kits or assays for determining target molecules. In one embodiment the test kit of the present invention is a one-step immunoassay kit and/or a FRET immunoassay kit. In a very specific embodiment, the test kit comprises reagents for performing said immunoassay or method of the present invention, and/or instructions for performing said immunoassay or method of the present invention, wherein optionally at least one or two recombinant antibodies or antigen binding fragments (such as one or two Fabs) are utilized.

For example, the kit for determining a target molecule such as Staphylococcus aureus Enterotoxin A and optionally comprising at least one antibody or antigen binding fragment of the present invention can be a kit for conventional ELISA, immunoPCR or FIA, e.g., a sandwich test e.g., in microtiter wells, or a sensor-based immunoassay. Actually, the kit may be for any assay types, such as lateral flow test, agglutination test, capillary electrophoresis, antibody arrays, sensor assays (e.g. SPR sensor assays), fusion proteins (such as alkaline phosphatase (AP))-based assays, fluorescent or magnetic bead-based assays and/or microfluidic assay systems, or any combination thereof.

Optionally control samples (e.g., positive or negative control samples) or reference levels revealing the presence, absence, or specific target molecule levels may also be utilized in the present invention. Also, a quality control may optionally be com-prised within the test kit.

For example, the reagents (e.g., at least antibody or antigen binding fragment) of the kit or used in the method of the present invention can be in a pre-dried form e.g., in the well of a microtiter plate. Then at least one sample or a dilution series of at least one sample can be added to said well. In such a case the test kit may comprise multiple reagent pairs physically separated from each other e.g., in the form of a microarray, whereby multiple antigens may be tested simultaneously from a sample. The test kit of the present invention may also be applied to high through-put methods for large number of samples. As used herein “a high-throughput” kit or method refers to a kit or method, wherein e.g. (liquid handling) devices and/or sensitive detectors are used to quickly conduct even up to thou-sands or millions of tests, wherein specifically complexes of one or more antibody or antigen binding fragments thereof and a target molecule are formed. Indeed, the test kits and methods of the present invention can include, but are not limited to, multiplex assays and methods e.g., utilizing different fluorescent and/or other labels (e.g., chemical labels, nanoparticles and/or quantum dots).

In a specific embodiment the present invention can be applied in the fast diagnostics of target molecules. As used herein “fast diagnostics” refers to at least immunoassays (such as ELISA), sensor solutions and similar applications known to a skilled person.

In a specific embodiment the method, assay, FRET pair, antibody or antigen binding fragment, or kit is for determining a target molecule, Staphylococcus aureus Enterotoxin or Staphylococcus aureus Enterotoxin A in a sample.

Furthermore, the present invention concerns use of the FRET pair, recombinant antibody or an antigen binding fragment, or kit for determining a target molecule such as Staphylococcus aureus enterotoxin or Staphylococcus aureus Enterotoxin A in a sample.

And still, the present invention concerns a method for determining Staphylococcus aureus (e.g. indirectly by determining enterotoxins of Staphylococcus aureus) or enterotoxins thereof such as Staphylococcus aureus Enterotoxin A. Said method is not limited to but can be selected e.g. from the group consisting of ELISA, immunoPCR or FIA. In one embodiment the method may be for example a conventional sandwich test e.g. in microtiter wells or a lateral flow-test. Furthermore, any other method or assay types, such as agglutination test, lateral flow test, capillary electrophoresis, antibody arrays, cantilivers and/or microfluidic assay systems, or any combination thereof can be applied in the present invention. Indeed, the method for determining the presence, absence or level (such as concentration) of SEA may comprise use of one or more of said (immune)assays. Optional washing steps (e.g., with a suitable buffer or water) can be applied in the method if needed. Determining or measuring the presence, absence, level or amount of SEA in a sample may be carried out e.g., based on the colorimetric, fluorescent, paramag-netic, electrochemical or label free (e.g., surface plasmon resonance and quartz crystal microbalance) detection mode. Optionally determination may also comprise use of any suitable statistical methods known to a person skilled in the art.

In one embodiment of the invention the presence, absence or levels of target molecules are determined in vitro and/or in situ from a sample. Indeed, the method, FRET pair, antibodies or antigen binding fragments, or kit are suitable for testing samples in laboratory setting or on-site i.e., in places elsewhere than the laboratory. In one embodiment of the invention the sample is a biological sample, environmental sample, process sample, (raw) material sample, food sample, sample of a food process, dairy product sample, sample of a biochemical agent, swab, or a sample of a subject. In one embodiment the sample has not been pretreated or has been pretreated before using according to the present invention or in the present invention. For example, extraction can be carried out for samples such as solid or semi-solid samples for obtaining pretreated samples. In one embodiment of the invention a subject is a human or an animal, a child, an adolescent or an adult. Also, any animal, such as a pet, domestic animal or production animal, may be a subject of the present invention. The sample obtained from a subject includes but is not limited to a body fluid sample or a sample of blood, serum, plasma, urine, feces, saliva, tears, sweat, secretion, biopsy or tissue, or a sample from vagina. Indeed, the method, FRET pair, antibodies or antigen binding fragments, or kit of the present invention may be employed for all kinds of investigations, such as in detecting toxic compounds in food, feed and in environment, proteins/molecules indicative of ongoing processes, contamination at surfaces and in clinical tests, and pharmacological research. Furthermore, in one embodiment one or more antibodies or antigen binding fragments thereof (e.g., a combination of two different antibodies and/or antigen binding fragments) of the present invention can be used as neutralizing antibodies in order to prevent a target molecule from acting as a superantigen.

The global food safety testing market can be categorized into meat and meat products; dairy and dairy products; cereal, grain, and pulse; processed food; and other ingredients. Among many other agents and products dairy products and milk serve as good substrates for the growth of bacteria such as S. aureus which can contaminate raw milk but also any phase in milk processing.

One or more antibodies or antigen binding fragments thereof of the present inven-tion can further be used for treating such as pretreating a sample or in a method of treating or pretreating a sample. In a method of treating or pretreating a sample said method comprises allowing at least one antibody or a fragment thereof to contact with a target molecule (such as one or more Staphylococcus aureus enterotoxins, e.g. Staphylococcus aureus Enterotoxin A) of a sample thereby forming a complex comprising said at least one antibody or antigen binding fragment thereof and the target molecule, and optionally separating, purifying and/or con-centrating the target molecule from the complex of a sample e.g. milk. In one embodiment the anti-SEA antibody or antigen binding fragment thereof can be used for sample preparation (such as purification and/or concentration of SEA or other SE's) in different sample preparation methods including but not limited to immunoaffinity columns, other immunoaffinity matrices, magnetic beads, tips, stir bars, needles and capillaries. Sample preparation based on anti-SEA antibodies or antigen binding fragments thereof can be a separate function prior to any analysis such as an analysis including but not limited to biosensors, ELISA, FRET, Lateral flow assay, Mass spectrometer and liquid chromatography. Alternatively, sample preparation can be directly conjugated to an immunoassay e.g. having one anti-SEA antibody (e.g. of the present invention) on magnetic beads performing the sample preparation while the other anti-SEA antibody (e.g. of the present inven-tion) is used for detection (e.g. using fluorescence, enzymatic and/or electrochemical method).

Herein, the terms “polypeptide” and “protein” are used interchangeably to refer to polymers of amino acids of any length.

As used herein “polynucleotide” refers to any polynucleotide, such as single or double-stranded DNA (genomic DNA or cDNA) or RNA, comprising a nucleic acid sequence encoding a polymer of amino acids or a polypeptide in question or a conservative sequence variant thereof. Conservative nucleotide sequence variants (i.e., nucleotide sequence modifications, which do not significantly alter biological properties of the encoded polypeptide) include variants arising from the degenera-tion of the genetic code and from silent mutations.

Minor variations or modifications of any one of the sequences or subsequences set forth in the description and claims are still within the scope of the invention optionally provided that they do not affect the binding activity and/or affinity of the antibody or antigen binding fragment. Methods for making any genetic modifications are generally well known and are described in various practical manuals describing laboratory molecular techniques.

It will be obvious to a person skilled in the art that, as the technology advances, the inventive concept can be implemented in various ways. The invention and its embodiments are not limited to the examples described below but may vary within the scope of the claims.

### EXAMPLES

### Example 1. Construction of the Staphylococcus aureus Specific Antibody Library

The antibody library was constructed from an immunized mouse as described previously by Tullila and Nevanen (2017, International Journal of Molecular Sciences 18(6), 1169). In short, total RNA was extracted from a homogenized mouse spleen followed by cDNA synthesis. Amplification of the antibody genes were performed with PCR (polymerase chain reaction) using specific primers for light (K-chain, domains V_{L}-C_{L}) and heavy (domains V_{H}-C_{H1}) chains of mouse IgG. Amplified light and heavy chains were cloned to a phagemid vector as a Fab-fragment fused to minor coat protein pill of M13 bacteriophage. Next, the Fab-fragments were displayed on bacteriophages containing the corresponding antibody gene. The amino acid sequences of 96 individual antibody clones were determined. The functional size of the library was 6 million different antibody genes after subtracting identical or antibody sequences with defects. The size and diversity of the SEA specific antibody library met the criteria to proceed to the selection of SEA specific antibody clones.

### Example 2. Selection of the Anti-SEA Antibodies

Streptavidin coated magnetic beads were functionalized with the biotinylated SEA antigen. KingFisher™ magnetic bead processor (Thermo Fisher Scientific) was used for the selection of the anti-SEA antibodies. The SEA coated magnetic beads were incubated with antibody phage display library, the unbound phages were washed away and the bound phages, carrying antibodies specific for SEA, were eluted and subjected for the next round of selection. In total three rounds of selection were performed. Two parallel approaches were applied for elution: acid (100 mM glycine-HCl, pH 1.5) or base (100 mM TEA (triethylamine), pH 11,6). Various elution conditions may lead to different antibodies as shown previously by Tullila and Nevanen (2017, International Journal of Molecular Sciences 18(6), 1169).

The outcome of the selection rounds was evaluated by phage-ELISA. 50 ng of the biotinylated SEA antigen was immobilized onto streptavidin-coated ELISA plate wells, washed, and the phage pools in dilution series (10¹¹-10⁷ phages/ml) in PBST buffer were added to the wells. After incubation of 1,5 h at room tempera-ture, the plate wells were washed again and the bound phages were detected by a detection antibody, (anti-M13-HRP conjugate and ABTS (27-Azino-bis(3-ethylbenzothiazoline-6-sulfonic acid) diammonium salt) substrate) supplemented with 0.05% H₂O₂ and the absorbance was read at 405 nm after 10 minutes.

### Example 3. Screening of the Anti-SEA Antibodies

93 single bacterial colonies carrying an antibody gene were picked from plates from the third round of selection and grown in 1 ml of Super Broth (SB)-ampicillin (100 μg/ml) −1% glucose medium over night before inoculating to fresh SB-ampicillin medium. IPTG (Isopropyl ThioGalactose) was used to induce the antibody production to the culture supernatant. Next day the cells were harvested by centrifugation. Supernatants containing antibodies were subjected to determination of SEA/SEB binding activity by ELISA.

Primary positive antibodies were detected for specificity to SEA. Anti-SEA antibod-ies showing highest variation in amino acid sequence were chosen for further characterization.

The binding properties of anti-SEA antibodies obtained from the primary screening were further studied by competitive ELISA where 1 and 10 nM concentrations of soluble SEA was used in competition. None of the SEA-antibodies showed any remarkable cross-reactivity with SEB (results not shown). The two antibodies (clones 55 and 90) had excellent binding properties (FIG. 6). Already 1 nM SEA shows inhibition of antibody binding to the SEA functionalized surface. These two antibodies were cloned to the VTT expression vector pKKTac as Fab-fragments containing a Hiss-tag for purification (Tullila and Nevanen 2017, International Journal of Molecular Sciences 18(6), 1169).

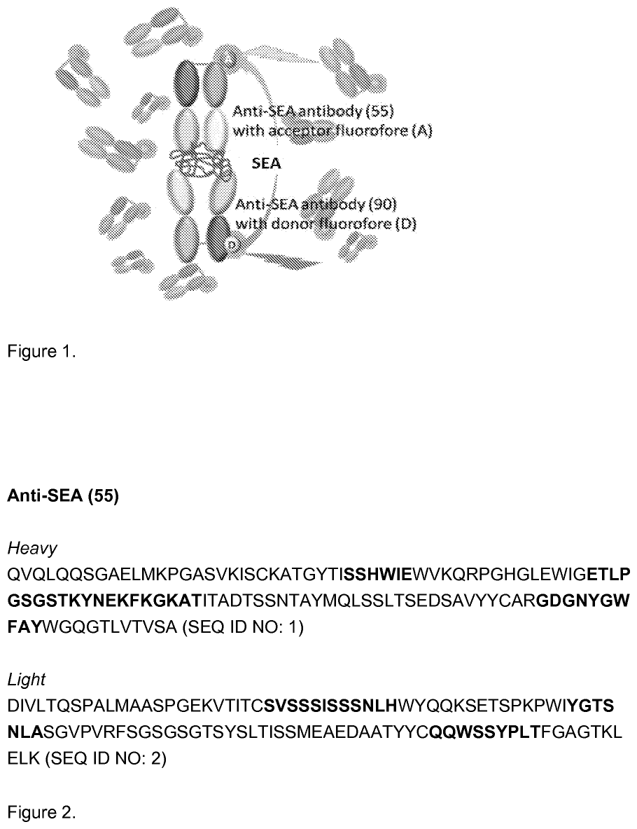

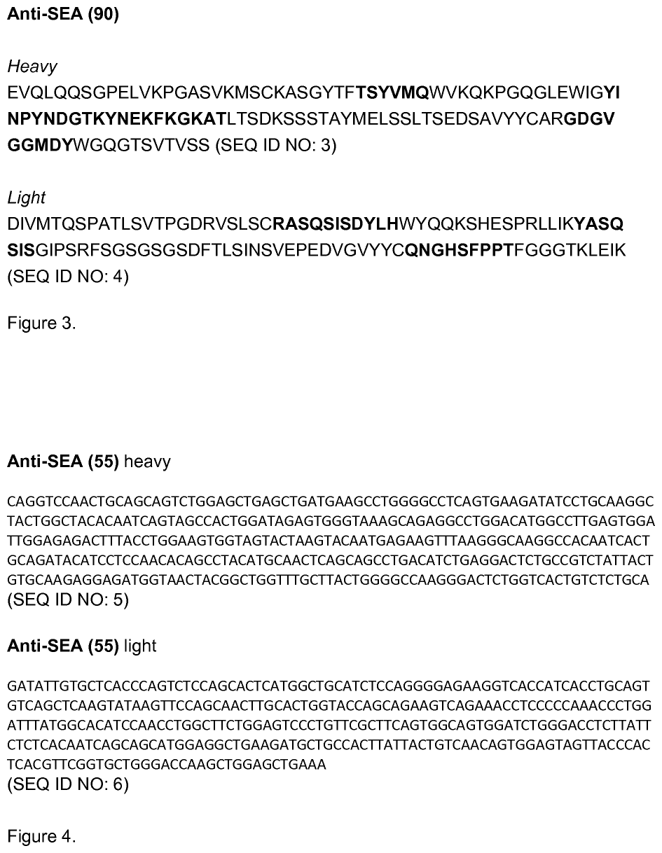

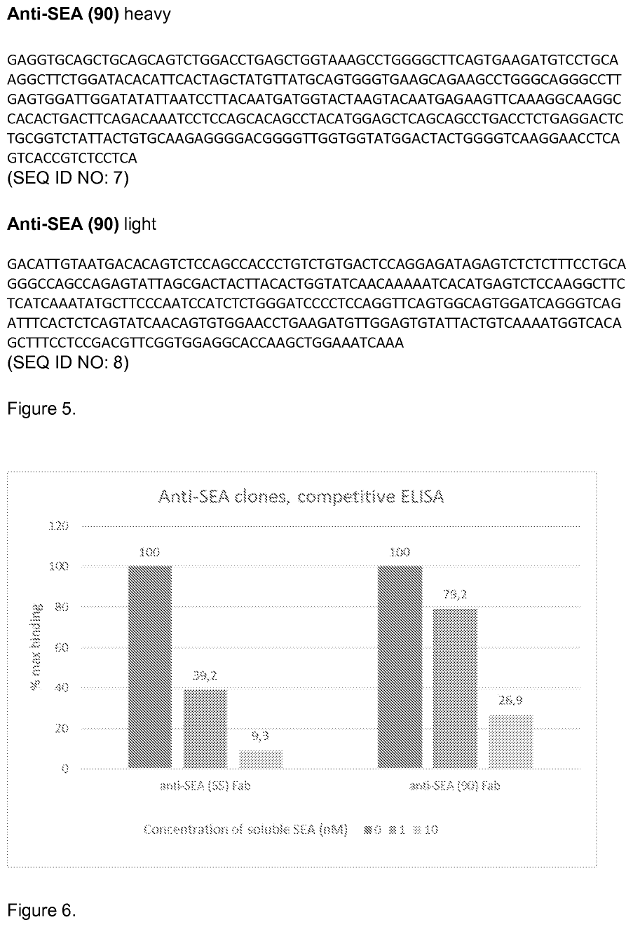

For the amino acid sequences of heavy and light chains of anti-SEA 55 and 90 see FIGS. 2 and 3 and SEQ ID NOs: 1, 2, 3 and 4. For the polynucleotide se-quence encoding the heavy and light chains of anti-SEA 55 and 90 see FIGS. 4 and 5 and SEQ ID NOs: 5, 6, 7, and 8.

### Example 4. Production and Purification of Anti-SEA

Anti-SEA antibodies ((55) and (90) Fabs) were produced in the RV308 E. coli strain using the pKKtac plasmid (Tullila and Nevanen 2017 International Journal of Molecular Sciences 18(6), 1169) and purified by metal affinity chromatography and protein G affinity chromatography according to the manufacturer's instructions (Cytiva lifesciences. Buffer was exchanged to PBS by dialysis before characterization of the binding properties of the antibodies. Purity of each of the proteins was evaluated with SDS-PAGE protein gel. Fab-fragments accounted for 88-96% of the proteins at SDS-PAGE measured by Molecular Imager® Gel Doc™XR+ and analysed with Image Lab 3.0. Software. Concentrations were calculated using theoretical extinction coefficients at 280 nm (ExPASy ProtParam tool) obtained from the sequencing data.

Subsets of strains containing antibodies or antigen binding fragments of the present invention (anti-SEA (55) Fab, anti-SEA (90) Fab) were deposited according to the Budapest Treaty on the International Recognition of the Deposit of Microorganisms for the Purposes of Patent Procedure, 1977, in VTT Culture Collection, P.O. Box 1000, FI-02044 VTT, FINLAND (http://culturecollection.vtt.fi), on Nov. 12th, 2020, with the following accession numbers, respectively: VTT E-203593 and VTT E-203594.

### Example 5. SPR (Biacore) Analysis

Characterization of the binding properties of anti-SEA antibodies was done by SPR analysis (Surface Plasmon Resonance). Using a Biacore T200 Cytiva lifesciences, a very sensitive SPR equipment, anti-SEA antibodies ((55) and (90) Fabs) were studied enabling the determination of the kinetic values, association and dissociation, for the binding. For this purpose, various sensor chips and reagents are commercially available.

Method Development

Biotinylated SEA was immobilized onto the commercial SA (Streptavidin)-CM5 chips (Cytiva lifesciences). The chip surfaces were conditioned by 3×1 min injec-tions of 1 M NaCl in 50 mM NaOH (flow rate 5 μl/ml). Biotinylated SEA (100 ng/ml, 16 min, 5 μl/min) was immobilized on active surface and 400 response units (RUs) were obtained. HBS-EP (1 x) was used as running buffer. Regeneration scouting for anti-SEA Fab 55 (500 nM) was done using 10 mM glycine buffers having the following pH values: 3.0, 2.5, 2.0 and 1.5. The results indicated that 10 mM glycine pH 2.5 was the best buffer as the surface showed consistent binding among 5 rep-licate cycles.

Affinity Determination

Concentration series from 0.24-1000 nM of anti-SEA (55) Fab and anti-SEA (90) Fab were applied to the SEA functionalized surface and the binding kinetics of the antibodies for SEA were determined. Plain SA surface was used as a reference.

Anti-SEA antibodies ((55) and (90) Fabs) had different binding kinetics. Table 1 illustrates the differences in association and dissociation of said anti-SEA antibod-ies.

TABLE 1
Summary of the binding kinetics and affinity constants
of anti-SEA anti-bodies (anti-SEA (55) and (90) Fabs)

| | anti-SEA (55) Fab | anti-SEA (90) Fab |
|---|---|---|
| Association rate constant | 9.7*10⁴ | 4.2*10⁵ |
| (ka, 1/Ms) | | |
| Dissociation rate constant | 3.5*10⁴ | 1.3*10⁻³ |
| (kd, 1/s) | | |
| Affinity constant (KD, M) | 3.6*10⁻⁹ | 3.0*10⁻⁹ |

Binding Site (Epitope) Analysis

Binding site analysis was performed by SPR (Biacore T200). The same biotinylated SEA surface as in kinetic analyses was used in epitope analysis.

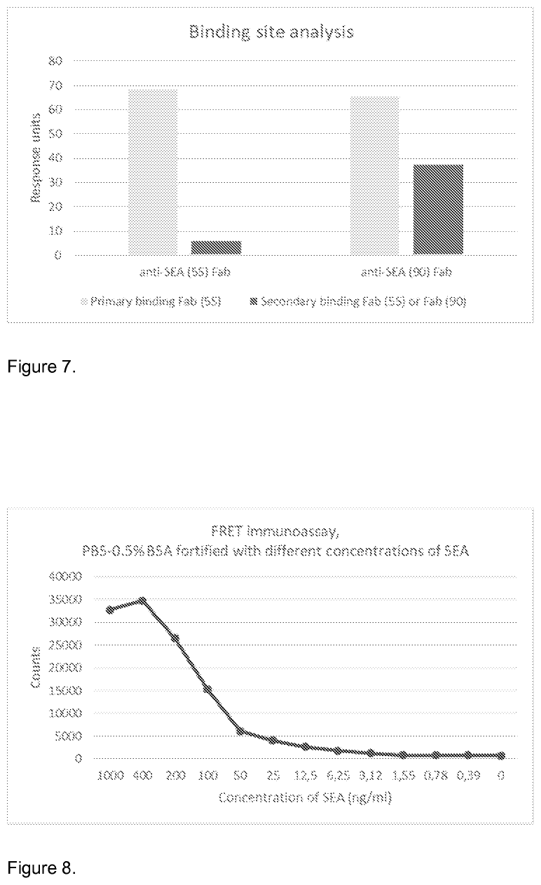

Anti-SEA (55) Fab was selected for the saturation antibody due to the slowest dissociation from the SEA surface. Anti-SEA (55) Fab at a 1 μM concentration was injected at a flow rate of 30 μl/min for 3 min to reach the saturation phase with maximum response for the binding. Then 250 nM anti-SEA (90) Fab was injected for 3 min at a flow rate of 30 μl/min to the anti-SEA (55) Fab saturated sensor surface. Anti-SEA (90) Fab shows a good additional response signal, indicating binding to a different epitope of SEA than anti-SEA (55) Fab. FIG. 7

### Example 6. A Method and Tools for Determining a Target Molecule SEA in a Sample by Utilizing Two Antibodies or Antigen Binding Fragments Capable of Generating a FRET Signal

The principle of the SEA FRET immunoassay is shown in FIG. 1. In presence of SEA in sample the fluorescent labelled anti-SEA antibodies bind to the close epitopes of SEA making possible the measurement of the FRET signal with fluorometer.

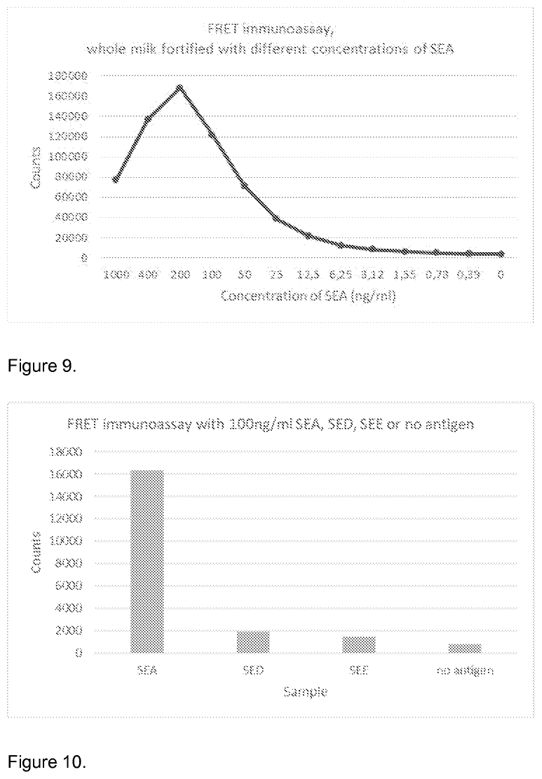

Europium-labelled anti-SEA (90) Fab and Alexa 647-labelled anti-SEA (55) Fab were used in concentrations of 5 and 10 μg/ml, respectively. 10 μl of each labelled anti-SEA antibodies were applied to the microtiterplate well. 80 μl SEA in concen-trations of 0-1000 ng/ml in PBS-0.5% BSA buffer or in whole milk (Homogenoimaton taysmaito, Valio) were applied and the FRET signal was measured with a fluorometer (Victor, Wallac-PerkinElmer). Two parallels for each SEA concentration was used and six parallels for Zero sample. Graphs are presented in FIGS. 8 and 9.

SEA FRET assay developed detected SEA at the <1 ng/ml in both buffer and milk spiked with SEA (FIGS. 8 and 9). Higher background binding was observed for milk.

### Example 7. Specificity of the Homogeneous FRET Assay for SEA

Staphylococcus aureus Enterotoxins D (SED) and E (SEE) share with SEA the amino acid sequence homology of 51% and 81%, respectively (Balaban and Rasooly, 2000, International Journal of Food Microbiology 61:1-10). SED and SEE were used to determine the specificity of the FRET immunoassay for SEA.

10 μl of Europium-labelled anti-SEA (90) Fab (5 μg/ml) and 10 μl of Alexa 647-labelled anti-SEA (55) Fab (10 μg/ml) were applied onto the microtiterplate well. 80 μl samples of SEA, SED or SEE in concentration of 100 ng/ml in PBS-0.5% BSA buffer were applied to the microtiterplate well and the FRET signal was measured with a fluorometer (Victor, Wallac-PerkinElmer). FRET immunoassay is specific for SEA. Samples containing 100 ng/ml SED or SEE produce no remarkable signals. Results are presented in FIG. 10.

In summary, a sensitive measurement of SEA by FRET immunoassay was shown for buffer and non-homogenised whole milk spiked with SEA. FRET immunoassay has high specificity for SEA.

### Example 8. A Luminescence Complementation Assay and Tools for Determining a Target Molecule SEA in a Sample by Utilizing Two Antibodies or Antigen Binding Fragments Fused with Peptides B9 and B10 Complementing the Truncated Nanoluc® Enzyme (Promega)

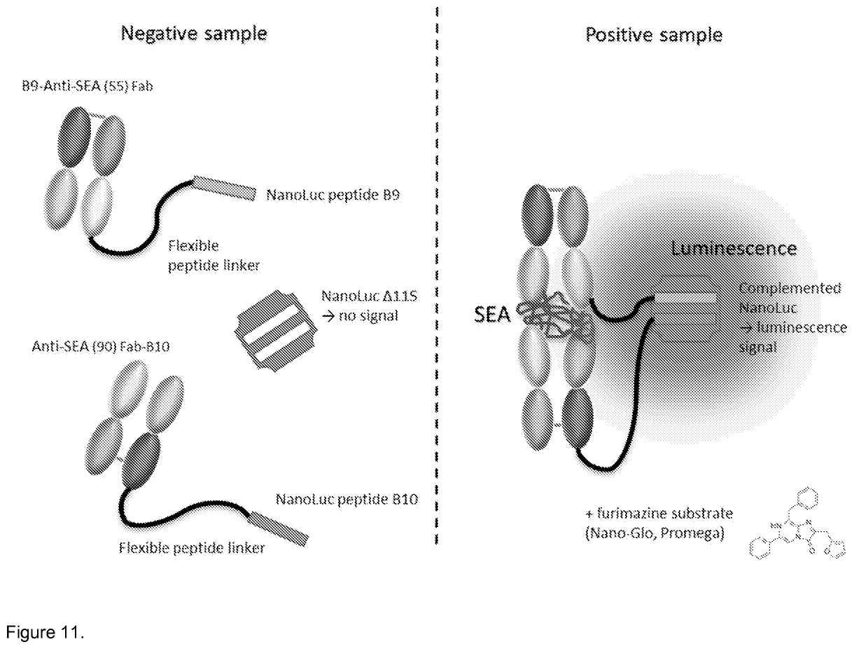

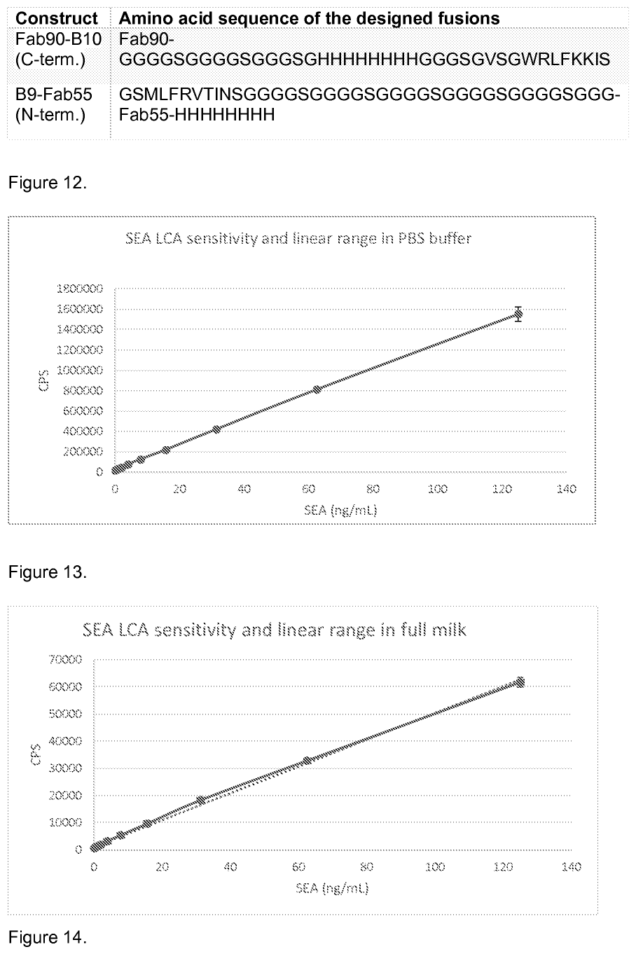

The principle of the luminescence complementation assay for SEA is shown in FIG. 11. Method utilizes anti-SEA (55) Fab (SEQ ID NO: 2) having N-terminal fusion with Nanoluc® peptide B9 (SEQ ID NO:9), anti-SEA (90) Fab (SEQ ID NO: 3) with C-terminal fusion with Nanoluc® peptide B10 (SEQ ID NO:10), and truncated Nanoluc® Δ11s-enzyme which is inactive without the peptides B9 and B10. Amino acid sequences of the linkers, His-tags for purification purposes and Nanoluc® peptides B9 and B10 are shown in FIG. 12. In presence of SEA in sample the anti-SEA antibodies bind to the ajacent epitopes of SEA bringing the B9 and B10 to close proximity allowing simultaneous complementation of the truncated NanoLuc® Δ11S present in reaction mixture. Luminescence signal can be read by after adding the furimazine substrate.

25 μl sample mix contained 20 nM anti-SEA (90) Fab-B10 and B9-anti-SEA (55) Fab, 200 nM of truncated NanoLuc® Δ11S and SEA in concentrations of 0-250 ng/ml in PBST buffer or in whole milk (Homogenoimaton taysmaito, Valio). Finally, 25 μl of furimazine substrate (1:250 dilution in PBS) (Nano-Glo, Promega) was added and the luminescent signal was measured with Envision multimode plate reader (Perkin Elmer) with luminescence 700 emission filter and luminescence 404 mirror using measurement height of 10 mm and measurement time 2 s. Six parallels for each SEA concentration and zero sample were used Graphs are presented in FIGS. 13 and 14.

Limit of detection for SEA was 0.26 and 0.5 ng/ml for fortified buffer and milk, respectively. Signal was stable 5-60 minutes.

1. A method for determining a target molecule in a sample, wherein the method comprises:
allowing a first recombinant antibody or antigen binding fragment thereof, which is capable of binding a target molecule, and a second recombinant antibody or antigen binding fragment thereof, which is capable of binding said target molecule at one or more different binding sites compared to the first recombinant antibody or antigen binding fragment thereof, to contact with a sample,
determining presence or absence or a level of the target molecule in the sample, wherein the first recombinant antibody and the second recombinant antibody are capable of generating a förster resonance energy transfer (FRET) reaction when binding the target molecule, and wherein the target molecule is Staphylococcus aureus Enterotoxin A,

2. The method of claim 1, wherein the method is a one-step immunoassay method.

3. The method of claim 1, wherein the first and second antibodies or antigen binding fragments thereof are allowed to contact with the sample simultaneously or sequentially.

4. A förster resonance energy transfer (FRET) pair comprising a first recombinant antibody or antigen binding fragment thereof that binds a target molecule and a second recombinant antibody or antigen binding fragment thereof that binds the target molecule at one or more different binding sites compared to the first recombinant antibody or antigen binding fragment thereof, wherein the target molecule is Staphylococcus aureus Enterotoxin A.

5. (canceled)

6. (canceled)

7. (canceled)

8. The FRET pair of claim 4, wherein the first antibody or antigen binding fragment comprises amino acids of one or more complementarity determining regions (CDRs) selected from the group consisting of
a light chain region 1 comprising amino acids 24-35 of SEQ ID NO: 2,
a light chain region 2 comprising amino acids 50-56 of SEQ ID NO: 2,
a light chain region 3 comprising amino acids 90-98 of SEQ ID NO: 2,
a heavy chain region 1 comprising amino acids 30-35 of SEQ ID NO: 1,
a heavy chain region 2 comprising amino acids 50-69 of SEQ ID NO: 1, and a heavy chain region 3 comprising amino acids 99-108 of SEQ ID NO: 1 or the second antibody or antigen binding fragment comprises amino acids of one or more complementary determining regions selected from the group consisting of:
a light chain region 1 comprising amino acids 24-34 of SEQ ID NO: 4,
a light chain region 2 comprising amino acids 50-56 of SEQ ID NO: 4,
a light chain region 3 comprising amino acids 89-97 of SEQ ID NO: 4,
a heavy chain region 1 comprising amino acids 30-35 of SEQ ID NO: 3,
a heavy chain region 2 comprising amino acids 50-69 of SEQ ID NO: 3, and
a heavy chain region 3 comprising amino acids 99-107 of SEQ ID NO: 3.

9. (canceled)

10. The method or FRET pair of claim 4, wherein the first antibody or an antigen binding fragment thereof comprises an amino acid sequence having at least 90% sequence identity to SEQ ID NO: 1 and/or SEQ ID NO: 2; or
the heavy chain of the first antibody or an antigen binding fragment thereof comprises SEQ ID NO: 1, and/or the light chain of the first antibody or an antigen binding fragment thereof comprises SEQ ID NO: 2 or
the second antibody or an antigen binding fragment thereof comprises an amino acid sequence having at least 90% sequence identity to SEQ ID NO: 3 and/or SEQ ID NO: 4; or
the heavy chain of the second antibody or an antigen binding fragment thereof comprises SEQ ID NO: 3, and/or the light chain of the second antibody or an antigen binding fragment thereof comprises SEQ ID NO: 4.

11. (canceled)

12. The method or The FRET pair of claim 4, wherein the antigen binding fragment of the first and/or second recombinant antibody is a single chain Fv (scFv) or Fab fragment.

13. A recombinant antibody or antigen binding fragment thereof that binds Staphylococcus aureus Enterotoxin A, wherein the antibody or antigen binding fragment thereof comprises amino acids of one or more complementarity determining regions (CDRs) selected from the group consisting of
a light chain region 1 comprising amino acids 24-35 of SEQ ID NO: 2,
a light chain region 2 comprising amino acids 50-56 of SEQ ID NO: 2,
a light chain region 3 comprising amino acids 90-98 of SEQ ID NO: 2,
a heavy chain region 1 comprising amino acids 30-35 of SEQ ID NO: 1,
a heavy chain region 2 comprising amino acids 50-69 of SEQ ID NO: 1, and
a heavy chain region 3 comprising amino acids 99-108 of SEQ ID NO: 1,
or the antigen binding fragment thereof comprises amino acids of one or more complementarity determining regions (CDRs) selected from the group consisting of
a light chain region 1 comprising amino acids 24-34 of SEQ ID NO: 4,
a light chain region 2 comprising amino acids 50-56 of SEQ ID NO: 4,
a light chain region 3 comprising amino acids 89-97 of SEQ ID NO: 4,
a heavy chain region 1 comprising amino acids 30-35 of SEQ ID NO: 3,
a heavy chain region 2 comprising amino acids 50-69 of SEQ ID NO: 3, and
a heavy chain region 3 comprising amino acids 99-107 of SEQ ID NO: 3.

14. (canceled)

15. The antibody or antigen binding fragment thereof of claim 13, wherein the antibody or an antigen binding fragment thereof comprises an amino acid sequence having at least 90% sequence identity to SEQ ID NO: 1 and/or SEQ ID NO: 2; or
the heavy chain of the antibody or an antigen binding fragment thereof comprises SEQ ID NO: 1, and/or the light chain of the antibody or an antigen binding fragment thereof comprises SEQ ID NO: 2 or
wherein the antibody or an antigen binding fragment thereof comprises an amino acid sequence having at least 90% sequence identity to SEQ ID NO: 3 and/or SEQ ID NO: 4; or
the heavy chain of the antibody or an antigen binding fragment thereof comprises SEQ ID NO: 3, and/or the light chain of the antibody or an antigen binding fragment thereof comprises SEQ ID NO: 4.

16. (canceled)

17. (canceled)

18. (canceled)

19. The antibody or antigen binding fragment thereof of claim 13, wherein the antigen binding fragment is a single chain Fv (scFv) or Fab fragment.

20. (canceled)

21. An isolated nucleic acid molecule comprising a nucleotide sequence that encodes the recombinant antibody or antigen binding fragment of claim 13.

22. The isolated nucleic acid molecule of claim 21 comprising a polynucleotide sequence having at least 90% sequence identity to SEQ ID NO: 5, 6, 7 and/or 8.

23. An expression vector comprising the nucleic acid molecule of claim 21.

24. (canceled)

25. A method for determining Staphylococcus aureus Enterotoxin A in a sample, wherein the method comprises allowing a recombinant antibody or antigen binding fragment of claim 13 to contact with a sample and thereafter determining the presence, absence or level of Staphylococcus aureus Enterotoxin A in the sample.

26. The method according to claim 1, wherein wherein the sample is a biological sample, environmental sample, process sample, (raw) material sample, food sample, sample of a food process, dairy product sample, sample of a biochemical agent, swab, or a sample of a subject.

27. (canceled)

28. (canceled)

29. A test kit comprising the FRET pair of claim 4, wherein the test kit is optionally a one-step immunoassay kit.

30. (canceled)

31. (canceled)

32. (canceled)

33. The method according to claim 1, wherein the first antibody or antigen binding fragment comprises amino acids of one or more complementarity determining regions (CDRs) selected from the group consisting of
a light chain region 1 comprising amino acids 24-35 of SEQ ID NO: 2,
a light chain region 2 comprising amino acids 50-56 of SEQ ID NO: 2,
a light chain region 3 comprising amino acids 90-98 of SEQ ID NO: 2,
a heavy chain region 1 comprising amino acids 30-35 of SEQ ID NO: 1,
a heavy chain region 2 comprising amino acids 50-69 of SEQ ID NO: 1, and a heavy chain region 3 comprising amino acids 99-108 of SEQ ID NO: 1 or the second antibody or antigen binding fragment comprises amino acids of one or more complementary determining regions selected from the group consisting of:
a light chain region 1 comprising amino acids 24-34 of SEQ ID NO: 4,
a light chain region 2 comprising amino acids 50-56 of SEQ ID NO: 4,
a light chain region 3 comprising amino acids 89-97 of SEQ ID NO: 4,
a heavy chain region 1 comprising amino acids 30-35 of SEQ ID NO: 3,
a heavy chain region 2 comprising amino acids 50-69 of SEQ ID NO: 3, and
a heavy chain region 3 comprising amino acids 99-107 of SEQ ID NO: 3.

34. The method according to claim 1, wherein the heavy chain of the first antibody or an antigen binding fragment thereof comprises SEQ ID NO: 1, and/or the light chain of the first antibody or an antigen binding fragment thereof comprises SEQ ID NO: 2 or the second antibody or an antigen binding fragment thereof comprises an amino acid sequence having at least 90% sequence identity to SEQ ID NO: 3 and/or SEQ ID NO: 4; or
the heavy chain of the second antibody or an antigen binding fragment thereof comprises SEQ ID NO: 3, and/or the light chain of the second antibody or an antigen binding fragment thereof comprises SEQ ID NO: 4.

35. The method of claim 1, wherein the antigen binding fragment of the first and/or second recombinant antibody is a single chain Fv (scFv) or Fab fragment.

36. The test kit according to 29, wherein the antigen binding fragment of the first and/or second recombinant antibody is a single chain Fv (scFv) or Fab fragment.

37. The method according to claim 25, wherein, wherein the sample is a biological sample, environmental sample, process sample, (raw) material sample, food sample, sample of a food process, dairy product sample, sample of a biochemical agent, swab, or a sample of a subject.


SEQUENCE LISTING (10 sequences)

SEQ ID NO: 1 - protein, 119 aa, Artificial sequence
  anti-SEA (55) Fab, heavy chain amino acid sequence
QVQLQQSGAELMKPGASVKISCKATGYTISSHWIEWVKQRPGHGLEWIGETLPGSGSTKYNEKFKGKATITADTSSNTAYMQLSSLTSEDSAVYYCARGDGNYGWFAYWGQGTLVTVSA

SEQ ID NO: 2 - protein, 108 aa, Artificial sequence
  anti-SEA (55) Fab, light chain amino acid sequence
DIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLELK

SEQ ID NO: 3 - protein, 118 aa, Artificial Sequence
  anti-SEA (90) Fab, heavy chain amino acid sequence
EVQLQQSGPELVKPGASVKMSCKASGYTFTSYVMQWVKQKPGQGLEWIGYINPYNDGTKYNEKFKGKATLTSDKSSSTAYMELSSLTSEDSAVYYCARGDGVGGMDYWGQGTSVTVSS

SEQ ID NO: 4 - protein, 107 aa, Artificial Sequence
  anti-SEA (90) Fab, light chain amino acid sequence
DIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIK

SEQ ID NO: 5 - DNA, 357 bp, Artificial Sequence
  anti-SEA (55) Fab, polynucleotide sequence encoding the heavy chain
caggtccaactgcagcagtctggagctgagctgatgaagcctggggcctcagtgaagatatcctgcaaggctactggctacacaatcagtagccactggatagagtgggtaaagcagaggcctggacatggccttgagtggattggagagactttacctggaagtggtagtactaagtacaatgagaagtttaagggcaaggccacaatcactgcagatacatcctccaacacagcctacatgcaactcagcagcctgacatctgaggactctgccgtctattactgtgcaagaggagatggtaactacggctggtttgcttactggggccaagggactctggtcactgtctctgca

SEQ ID NO: 6 - DNA, 324 bp, Artificial Sequence
  anti-SEA (55) Fab, polynucleotide sequence encoding the light chain
gatattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaa

SEQ ID NO: 7 - DNA, 354 bp, Artificial Sequence
  anti-SEA (90) Fab, polynucleotide sequence encoding the heavy chain
gaggtgcagctgcagcagtctggacctgagctggtaaagcctggggcttcagtgaagatgtcctgcaaggcttctggatacacattcactagctatgttatgcagtgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagaggggacggggttggtggtatggactactggggtcaaggaacctcagtcaccgtctcctca

SEQ ID NO: 8 - DNA, 321 bp, Artificial Sequence
  anti-SEA (90) Fab, polynucleotide sequence encoding the light chain
gacattgtaatgacacagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaa

SEQ ID NO: 9 - protein, 147 aa, Artificial sequence
  Fab90-B10 construct
DIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLELKGGGGSGGGGSGGGSGHHHHHHHHGGGSGVSGWRLFKKIS

SEQ ID NO: 10 - protein, 165 aa, Artificial sequence
  B9-Fab55 construct
GSMLFRVTINSGGGGSGGGGSGGGGSGGGGSGGGGSGGGEVQLQQSGPELVKPGASVKMSCKASGYTFTSYVMQWVKQKPGQGLEWIGYINPYNDGTKYNEKFKGKATLTSDKSSSTAYMELSSLTSEDSAVYYCARGDGVGGMDYWGQGTSVTVSSHHHHHHHH